(19) United States
(12) Patent Application Publication
(10) Pub. No.: US 2011/0045076 A1
(43) Pub. Date: Feb. 24, 2011

(54) LINEAR ORDER RELEASE POLYMER

(76) Inventors: Patrick F. Kiser, Salt Lake City, UT (US); Kavita Gupta, Salt Lake City, UT (US)

Correspondence Address: FOLEY & LARDNER LLP, 150 EAST GILMAN STREET, P.O. BOX 1497, MADISON, WI, 53701-1497, US

(21) Appl. No.: 12/864,168

(22) PCT Filed: Jan. 23, 2009
(86) PCT No.: PCT/US2009/031877

§ 371 Date: Nov. 15, 2010

Related U.S. Application Data
(60) Provisional application No. 61/023,716, filed on Jan. 25, 2008.

(51) Int. Cl. A61K 9/14; A61P 31/16; A61P 35/00; A61P 5/30; A61K 31/444; A61K 31/675; A61K 31/513; A61K 31/496; A61K 31/57

(52) U.S. Cl. 424/486; 514/334; 514/81; 514/274; 514/253.04; 514/178

(57) ABSTRACT

Intravaginal drug delivery devices, including intravaginal rings, are provided herein. The devices comprise a polyether urethane composition and a pharmaceutically effective amount of at least one vaginally administrable drug homogeneously distributed throughout the polyether urethane. The devices are capable of exhibiting a substantially zero order release profile of drug over extended periods of time. Also disclosed are methods for making the devices and methods of using the devices to prevent or treat a biological condition.

### FIELD OF THE INVENTION

The invention generally relates to intravaginal drug delivery devices. More specifically, intravaginal devices are disclosed which are capable of providing a zero order release of loaded drugs over extended periods of time. Methods of making and using the devices are also disclosed.

### BACKGROUND OF THE INVENTION

Intravaginal drug delivery devices, including intravaginal rings (IVRs), are typically formed from biocompatible polymers and contain a drug released by diffusion through the polymer matrix. The devices may be inserted into the vaginal cavity and the drug may be absorbed by the surrounding body fluid through the vaginal tissue. In some IVR designs, the drug is uniformly dispersed or dissolved throughout the polymer matrix (monolithic system). In other designs, the drug may be confined to an inner core within the ring (reservoir system). Monolithic systems are expected to show a diffusion-controlled square root of time release profile of the drug.^{10,24} Reservoir systems may exhibit a zero order release of loaded drugs, but the systems generally involve costly fabrication schemes.

To date, poly(ethylene-co-vinyl acetate), or EVA (for e.g. in NuvaRing®^{8,9}), and poly(dimethyl siloxane), or silicone (for e.g. in Estring³⁰, Femring® and in Population Councils progesterone-releasing ring³¹), are currently the only polymers commercially exploited for IVRs. Compared to thermoplastics, Sn-catalysed condensation-cured silicone is limited by a lower mechanical stiffness.³² Therefore, silicone IVRs are fabricated with larger cross-sectional diameters to achieve the retractive forces required for retention in the vaginal cavity, which may affect ring user acceptability. Moreover, the manufacturing costs associated with these IVRs are considerable.

### SUMMARY

The present invention provides intravaginal drug delivery devices, including intravaginal rings, methods for making the devices, and methods of using the devices. The devices comprise a polyether urethane composition and a pharmaceutically effective amount of at least one vaginally administrable drug homogeneously distributed throughout the polyether urethane composition. The present invention encompasses a broad range of intravaginal devices, including intravaginal rings.

The devices may comprise a variety of polyether urethanes or combinations of polyether urethanes. In some embodiments, the polyether urethane composition comprises a Tecoflex® polyurethane. In other embodiments, the polyether urethane composition comprises Tecoflex® EG-80A polyurethane.

Similarly, the devices may comprise a variety of vaginally administrable drugs or combinations of drugs. In some embodiments, the drug is selected from microbicides, contraceptive agents, hormones, estrogen receptor modulators, post-menopausal hormones, antiviral agents, anticancer agents and therapeutic peptides and proteins. In some embodiments, the drug is a microbicide and the microbicide is an anti-HIV agent or an anti-HPV agent. In some embodiments, the anti-HIV agent is selected from non-nucleoside reverse transcriptase inhibitors, including dapivirine, nucleoside reverse transcriptase inhibitors, including Tenofovir, and HIV entry inhibitors.

The devices comprise a pharmaceutically effective amount of the one or more vaginally administrable drugs. In some embodiments, the drug is present in an amount ranging from about 2 mg to about 60 mg of drug per gram of polyether urethane. In other embodiments, the drug is present in an amount ranging from about 0.1% w/w to about 10% w/w, where w/w refers to the weight ratio of the drug to the polyether urethane.

The devices of the present invention may further comprise a variety of additional components. In some embodiments, the devices comprise polyethylene glycol incorporated into the polyether urethane matrix. In some embodiments, the polyethylene glycol is present in an amount ranging from about 5% w/w to about 15% w/w, where w/w refers to the weight ratio of the polyethylene glycol to the polyether urethane.

The devices disclosed herein are capable of exhibiting a substantially zero order release profile of the drug over a period of at least one day. In some embodiments, the device exhibits a release rate of drug ranging from about 55 μg of drug per day to about 550 μg is of drug per day.

The present invention also provides methods of making the intravaginal devices disclosed herein. The methods comprise forming a drug-loaded polyether urethane composition into a shape suitable for use in intravaginal drug delivery, wherein a pharmaceutically effective amount of at least one vaginally administrable drug is homogeneously distributed throughout the composition. In some embodiments, the methods further comprise preparing the drug-loaded polyether urethane composition by dissolving at least one polyether urethane and at least one vaginally administrable drug in a solvent, and removing the solvent. In other embodiments, the methods further comprise preparing the drug-loaded polyether urethane composition by melting at least one polyether urethane to form a polyether urethane melt, and mixing at least one vaginally administrable drug into the polyether urethane melt. In embodiments in which the device is an intravaginal ring, the formation of the composition into the shape of the device comprises extruding the drug-loaded polyether urethane composition into a rod and joining the ends of the rod to form a ring.

Methods of using the intravaginal devices are also provided. The methods comprise releasing the drug from the device while the device resides in a subject's vagina. Any of the intravaginal devices disclosed herein may be used in such methods.

### BRIEF DESCRIPTION OF THE DRAWINGS

FIG. 1 collects differential scanning calorimetric (DSC) spectra for dapivirine, PU (Tecoflex® EG-80A) and dapivirine loaded PU.

FIG. 2 shows the results of extraction tests. Extracted dapivirine contents were not significantly different after extrusion compared to those prior to extrusions. SC and MM refer to the drug incorporated PU obtained by solvent casting and melt mixing, respectively.

FIG. 3 shows the release kinetics from PU-SC rods of various dapivirine loadings in 25:75 v/v isopropanol (i-prOH):water (5 mL) release medium.

FIG. 4 shows the cumulative release profiles obtained by integration of plots shown in FIG. 3. Note the linearity of cumulative amount of dapivirine released with time and absence of burst release (n=3, mean±S.D.).

FIG. 5 shows dapivirine flux from PU-SC rods vs. loading using 25:75 i-prOH:water as sink condition at 37° C. Note that the release rate is directly proportional to the dapivirine loading (n=3, mean±S.D.).

FIG. 6 shows the cumulative release of dapivirine linear with time from ring (SC) prototype, fabricated with a cross-sectional diameter of 4.4±0.1 mm and ID of 54 mm, using 25:75 i-prOH:water as sink condition at 37° C. (n=3, mean±S.D.).

FIG. 7 shows the cumulative release of dapivirine linear with time from ring (MM) prototype, fabricated with a cross-sectional diameter of 4.4±0.1 mm and ID of 54 mm, using 25:75 i-prOH:water as sink condition at 37° C. (n=3, mean±S.D.).

FIG. 8 demonstrates that the average dapivirine flux obtained from rings (SC) compared to those from rods (SC) for a month in 25:75 i-prOH:water as sink condition at 37° C. were 12% and 18% higher in 20 mg/g and 2.5 mg/g dapivirine loadings respectively (n=3, mean±S.D.).

FIG. 9 shows the cumulative flux of dapivirine from extruded PU rods (dapivirine loading in PU=5 mg/g) into liposome dispersions ([liposome]=10 mg/mL, osmolarity adjusted to 310 mOsm/kg with NaCl) in comparison to 25:75 i-prOH:water (n=3, mean±SD).

FIG. 10 shows the cumulative flux of model compound of Tenofovir (50 mg/g) from PEG incorporated PU (PEG/PU=5% w/w).

FIG. 11 illustrates a design for an IVR as disclosed herein.

FIG. 12 illustrates another design for an IVR as disclosed herein.

FIG. 13 illustrates the cumulative flux of Tenofovir loaded Tecophilic matrices ([Tenofovir]=50 mg/g) extruded into a solid cross-sectional rod.

FIG. 14 demonstrates the cumulative flux of Tenofovir ([Tenofovir]=50 mg/g) from Tecoflex EG-80A and EG-85A matrices with and without the incorporation of PEG ([PEG]=5% w/w).

FIG. 15 demonstrates the cumulative flux of Tenofovir ([Tenofovir]=50 mg/g) from Tecoflex EG-93A matrices incorporated with PEG ([PEG]=5% w/w) with and without the presence of salts such as NaCl ([NaCl]=2.5% w/w) and CaCl₂ ([CaCl₂]=2.5% w/w).

FIG. 16 shows the cumulative release of the antiviral drug SJ-3991 (1-(cyclopropylmethyl)-6-(3,5-dimethylbenzoyl)-5-isopropylpyrimidine-2,4(1H,3H)-dione) from Tecoflex EG-80A, Tecoflex EG-93A, and Tecophilic HP-60D-20 matrices.

### DETAILED DESCRIPTION

Intravaginal drug delivery devices, including intravaginal rings, are provided herein. Also provided are methods for making the devices and methods of using the devices to prevent or treat a biological condition. The devices comprise a polyether urethane composition and a pharmaceutically effective amount of at least one vaginally administrable drug homogeneously distributed throughout the composition. The inventors have made the surprising and unexpected discovery that the monolithic devices of the present invention are capable of releasing drugs in a zero order profile over extended periods of time. Thus, the devices do not require a drug reservoir and provide a cost-effective, patient compliant means of providing a sustained delivery of a variety of drugs, including drugs that prevent the transmission of HIV.

The intravaginal devices of the present invention comprise a polyether urethane composition and may be monolithic. Polyurethanes used in the present devices offer control of processing temperature, mechanical properties and drug release by modifying their components and ratios. The presence of a microphase separation leading to hard and soft domains imparts flexibility and strength to the polymer. Furthermore, polyether urethanes composed of a polymeric diol and short chain diol connected by urethane linkages through diisocyanates are practically non-degradable up to three years.

A variety of medical grade polyether urethanes may be used. For example, in some embodiments, the polyether urethanes are the reaction product of a polymeric diol, a short chain diol, and a diisocyanate. Diisocyanates include, but are not limited to, symmetrical molecules like methylene-bis-cyclohexyl diisocyanates, 1,4 cyclohexyl diisocyanate, and dicyclohexyl methane diisocyanate (HMDI). Short chain diols include, but are not limited to, 1,4 butane diol or similar symmetrical diols or assymetrical diols like 1,2 propane diol. Polymeric diols include, but are not limited to, poly tetra methylene ether glycol (PTMEG) chosen from a molecular weight of 500 to 10,000. In some embodiments, the polyether urethane comprises the reaction product of dicyclohexyl methane diisocyanate, a PTMEG having a molecular weight of between about 500 and about 10,000, and 1,4 butane diol. In other embodiments, the PTMEG has a molecular weight of about 1,000 to about 2,000. In some embodiments, the number of moles of dicyclohexyl methane diisocyanate is equal to the sum of the number of moles of PTMEG and the number of moles of 1,4 butane diol and the molar ratio of 1,4 butane diol to PTMEG is between about 1 to 1 and about 1.5 to 0.5. In some embodiments, the polyurethane has an average molecular weight of about 120,000 to about 180,000 and a weight average molecular weight of about 285,000 to about 335,000. These polyurethanes and their synthesis are described in detail in U.S. Pat. No. 4,523,005, which is hereby incorporated by reference in its entirety. These polyurethanes are also commercially available as the Tecoflex® family of polyurethanes manufactured by Lubrizol. Tecoflex® is a family of aliphatic polyether-based polyurethanes manufactured in several grades including EG-80A, EG-85A, EG-93A, EG-100A, EG-60D, EG-65D, EG-68D and EG-72D. The EG-80A and EG-85A polyurethanes use a PTMEG-2000 molecular weight polyol component while the EG-93A, EG-100A, EG-60D, EG-651D, EG-68D and EG-72D polyurethanes use a PTMEG-1000 molecular weight polyol component. In addition, the ratio of the short-chain diol to the polymeric diol differs in order to vary the hardness of each grade of polyurethane.

Other polyether urethanes include, but are not limited to, the Tecophilic®, and Tecothane® family of polyurethanes manufactured by Lubrizol. Tecophilic® is a family of aliphatic polyether-based polyurethanes, including hydrophilic polyether urethanes, manufactured in several grades including HP-60D-60, HP-60D-35, HP-60D-20, and HP-03A-100. Tecothane® is a family of aromatic polyether-based polyurethanes manufactured in several grades including TT-1074A, TT-1085A, TT-1095A, TT-1055D, TT-1065D, and TT-1075D-M. Any of the polyether urethanes described above may be used alone or in combination to form the intravaginal devices disclosed herein.

The intravaginal devices of the present invention also comprise a pharmaceutically effective amount of at least one vaginally administrable drug homogeneously distributed throughout the polyether urethane composition. In some embodiments, two drugs are homogeneously distributed throughout the polyether urethane composition. A variety of drugs may be used, alone or in combination, including, but not limited to microbicides, contraceptive agents, hormones, estrogen receptor modulators, post-menopausal hormones, antiviral agents and anticancer agents and therapeutic peptides and proteins. Representative microbicides, contraceptive agents and post-menopausal hormones include, but are not limited to, those disclosed in U.S. Pat. Nos. 4,292,965; 6,126,958; 6,476,079; and 6,951,654. Representative hormones include, but are not limited to gonadatropin releasing hormone agonists and leuprolide acetate. Contraceptive agents include, but are not limited to, 17a-ethinyl-levongestrel-17b-hydroxy-estra-4,9,11-trien-3-one, estradiol, etono-progestin alonegestrel, levongestrel, medroxyprogesterone acetate, nestorone, norethindrone, norgestrienone, progesterone, RU-486, etonogestril (3-keto-desogestrel), progestin, megestrol, 17-acetoxy-16-methylene-19-norprogesterone, and nestorone. Non-limiting examples of antiviral agents are provided in Example 10.

Representative estrogen receptor modulators include, but are not limited to, afimoxifene (4-hydroxytamoxifen), arzoxifene, bazedoxifene, clomifene, femarelle (DT56a), lasofoxifene, ormeloxifene, raloxifene, tamoxifen, toremifene, mifepristone (RU486), VA2914, ulipristal, Proellex, Asoprisnil, and CDB-4124.

In some embodiments, the microbicide is an anti-HIV agent or an anti-HPV agent. Anti-HIV agents include, but are not limited to, AMD-3100, BMS-806, BMS-793, C31G, carrageenan, CD4-IgG2, cellulose acetate phthalate, cellulose sulphate, cyclodextrins, Dextrin-2-sulphate, efavirenz, Etravirine (TMC-125), mAb 2G12, mAb b12, Merck 167, nonoxynol-9, plant lectins, poly naphthalene sulfate, poly sulpho-styrene, PRO2000, PSC-Rantes, Rilpivirine (TMC-278), SCH-C, SCH-D, T-20, TMC-125, UC-781, UK-427, UK-857, and Viramune. Anti-HPV agents include, but are not limited to pyrrole polyamides and lopinavir. The microbicide may also be an anti-HSV agents, including, but not limited to acyclovir, gangcyclovir, valacyclovir, and famciclovir.

Other anti-HIV agents may be used. By way of example only, agents that inhibit HIV-1 reverse transcriptase (RT), a key player in viral replication and establishment of infection in susceptible host cells, may be useful in curtailing HIV-1 transmission.¹⁴ Small molecular weight hydrophobic non-nucleoside RT inhibitors (NNRTI) with low systemic bioavailability and therefore longer retention in the vaginal tissue are ideal candidates far delivery through the disclosed devices.^{6,10,11,18,19} Dapivirine (formerly known as TMC120, also known as 4-[[4-[(2,4,6-trimethylphenyl)amino]pyrimidin-2-yl]amino]benzonitrile) is a specific and potent inhibitor of HIV-1 RT.^{6,11,18} Dapivirine is one of the diarylpyrimidine (DAPY) analogues which can adapt to structural changes in the NNRTI binding pocket in HIV-1 reverse transcriptase and therefore provide efficacy against a wide range of wild and resistant viruses at nanomolar concentrations.²⁰ The lipophilic NNRTI's are thought to partition into the lipid bilayer of cells, where they are retained there and gain entry into the hydrophobic core of the virus during the fusion of virus-cell membrane.²¹ Nucleoside reverse transcriptase inhibitors are another example of useful anti-HIV agents. Tenofovir (({[(2R)-1-(6-amino-9H-purin-9-yl)propan-2-yl]oxy}methyl)phosphonic acid) is a non-limiting example of a nucleoside reverse transcriptase inhibitor. As another non-limiting example, HIV entry inhibitors may be used as anti-HIV agents.

Other vaginally administrable drugs include Lidocaine, a cervical anaesthetic; Terbutaline, for dysmenorrhea and endometriosis; Sildenafil, for increased bloodflow to the uterus in preparation for embryo implantation; Misoprostol, for the induction of labor, cervical ripening, and pregnancy termination; Oxybutynin, for overactive bladder; Indomethacin, for the treatment of preterm labor; Bromocriptine, for the treatment of prolactinoma in those intolerant of nausea/vomiting side effects. Yet other vaginally administrable drugs include agents to treat bacterial vaginosis, antibacterial agents, and clindamycin.

The devices of the present invention comprise a pharmaceutically effective amount of one or more vaginally administrable drugs. By “pharmaceutically effective,” it is meant an amount which is sufficient to effect the desired physiological or pharmacological change in the subject. This amount will vary depending upon such factors as the potency of the particular drug, the desired physiological or pharmacological effect, and the time span of the intended treatment. Those skilled in the pharmaceutical arts will be able to determine the pharmaceutically effective amount for any given drug in accordance with standard procedures. In some embodiments, the drug is present in an amount ranging from about 2 mg to about 60 mg of drug per gram of polyether urethane. This includes embodiments in which the amount ranges from about 5 mg to about 50 mg, from about 10 mg to about 40 mg, and from about 20 mg to about 30 mg of drug per gram of polyether urethane. In some embodiments, the drug is Tenofovir and is present in an amount of about 50 mg per gram of polyether urethane. In other embodiments, the drug is dapivirine and is present in an amount ranging from about 2.5 mg to about 25 mg per gram of Tecoflex® EG-80A. This includes embodiments in which the amount ranges from about 3 mg to about 20 mg, from about 5 mg to about 18 mg, and from about 10 mg to about 15 mg per gram of Tecoflex® EG-80A. In other embodiments, the drug is present in an amount of about 0.1% w/w to about 10% w/w, where w/w refers to the weight ratio of the drug to the polyether urethane. This includes embodiments in which the amount ranges from about 0.2% w/w to about 8% w/w, from about 0.5% w/w to about 6% w/w, from about 1% w/w to about 5% w/w, and from about 2% w/w to about 4% w/w.

The intravaginal devices of the present invention may further comprise additional components, including, but not limited to, other polymers or pharmaceutically compatible agents. In some embodiments, the devices further comprise polyethylene glycol (PEG). In such embodiments, PEG may be present at varying amounts, including, but not limited to, amounts ranging from about 5% w/w to about 15% w/w, where w/w refers to the weight ratio of PEG to the polyether urethane. This includes embodiments in which the amount ranges from about 7% w/w to about 13% w/w and from about 9% w/w to about 11% w/w. In some embodiments, the polyether urethane is Tecoflex® EG-80A. A variety of pharmaceutically compatible agents may be used, including, but not limited to, those disclosed in U.S. Pat. No. 6,951,654.

The intravaginal devices of the present invention are capable of providing sustained delivery of one or more vaginally administrable drugs in a substantially zero order release profile. By substantially zero order it is meant that a substantially constant amount of drug is released over a given period of time. Because monolithic systems (i.e. devices in which the drug is present uniformly and homogenously throughout the device) are expected to show a diffusion-controlled square root of time release profile,^{10,24} the zero order release profiles exhibited by the devices of the present invention are both surprising and unexpected. In some embodiments, the devices exhibit a substantially zero order release profile of the drug over at least one day. In other embodiments, the devices exhibit a substantially zero order release profile of the drug over several days, over one month, or over more than a month. The release rate of drug from the devices of the present invention may be modified by changing the initial loading of the polyether urethane matrix with drug or by modifying the components or composition of the polyether urethane to make the polymer more or less hydrophobic. In some embodiments, the devices exhibit release rates ranging from about 55 μg of drug per day to about 550 μg of drug per day. This release rate is expected to be sufficient to achieve the desired therapeutic concentration of anti-HIV agents, including dapivirine, in the vagina to prevent sexual transmission of HIV.^{11,18} For example, when tested in clinical trials, silicone intravaginal rings that demonstrated a release rate of 50 μg of dapivirine per day in 50:50 v/v i-prOH:water maintained concentrations in the vaginal tissue 4-6 orders of magnitude higher than the required inhibitory concentrations.^{22,23} In other embodiments, the devices exhibit release rates ranging from about 60 μg of drug per day to about 500 μg of drug per day, from about 70 μg of drug per day to about 400 μg of drug per day, from about 80 μg of drug per day to about 200 μg of drug per day, and from about 90 μg of drug per day to about 150 μg of drug per day.

The intravaginal devices of the present invention may encompass a variety of shapes and sizes provided the device is compatible with vaginal administration to the subject and with the requirements imposed by drug delivery kinetics. In some embodiments, the device is in the form of an intravaginal ring (IVR). The dimensions of the IVR may vary depending upon the anatomy of the subject, the amount of drug to be delivered to the patient, the time over which the drug is to be delivered, the diffusion characteristics of the drug and other manufacturing considerations. The IVR should be flexible enough to enable bending and insertion inside the vaginal cavity and rigid enough to withstand the expulsive forces of the vaginal musculature^{26,27} without causing abrasion to the vaginal epithelium^{28,29}. In some embodiments, the inner diameter of the IVRs may range, e.g., from about 45 mm to about 65 mm. The cross-sectional diameter of the IVRs may range, e.g., from about 4 mm to about 10 mm. Other IVR designs are described in Example 6. Other intravaginal devices include tablets, pessaries, rods and films for adhesion to the mucosal epithelium as disclosed in U.S. Pat. No. 6,951,654.

The present invention also provides methods of making the intravaginal devices disclosed herein. The methods involve forming a drug-loaded polyether urethane composition into a shape suitable for use in intravaginal drug delivery, wherein a pharmaceutically effective amount of at least one vaginally administrable drug is homogeneously distributed throughout the composition. Drug reservoirs are not required and are typically excluded from the devices disclosed herein. In some embodiments, the methods further comprise preparing the drug-loaded polyether urethane composition by dissolving at least one polyether urethane and at least one vaginally administrable drug in one or more solvents, and removing the solvent. A variety of solvents or combinations of solvents may be used, including, but not limited to, dichloromethane, tetrahydrofuran, dimethylacetamide, and methylene chloride. The solvent may be removed by evaporation. In other embodiments, the methods further comprise preparing the drug-loaded polyether urethane composition by melting at least one polyether urethane to form a polyether urethane melt, and mixing at least one vaginally administrable drug into the polyether urethane melt. Each of these steps may be carried out under a variety of conditions, including, but not limited to those described in Example 1.

In some embodiments in which the device is an intravaginal ring, the step of forming the drug-loaded polyether urethane composition into the shape of the device involves extruding the drug-loaded polyether urethane composition into a rod and joining the ends of the extruded rod to form a ring. The ends of the ring may be joined together via a variety of biocompatible adhesives, including, but not limited to molten drug-free or drug-loaded polyether urethane. Each of these steps may be carried out under a variety of conditions, including, but not limited to those described in Example 1.

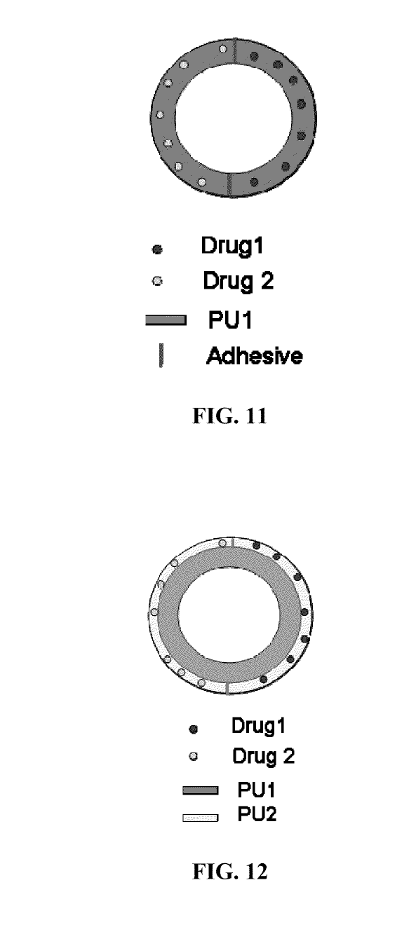

The methods disclosed herein may be adjusted as necessary to form the IVRs shown in FIGS. 11 and 12. For example, as shown in FIG. 11, two or more drug-loaded polyether urethane compositions may be extruded into two or more rods. The drug and polyether urethane of one rod may be the same or different as the drug and polyether urethane of another rod. The ends of the rods may be joined together to form a single ring. Alternatively, the rods may be divided into segments and the ends of the segments may joined together to form a single ring. Rods or segments of rods substantially free from drug may also be included into the ring. In another example shown in FIG. 12, a substantially drug-free polyether urethane composition formed into an inner ring. One or more drug-loaded polyether urethane compositions may be prepared and used to form a layer disposed around the inner ring. The polyether urethane of the inner ring may be the same or different as the polyether urethane used to form the outer layer. However, it will be clear to the skilled artisan that intravaginal devices disclosed herein, such as the disclosed IVRs, do not require a second layer of polymer to provide zero-order release of drug from the first layer.

The present invention further provides methods of using the intravaginal devices disclosed herein. The methods comprise releasing the drug from any of the intravaginal devices disclosed herein while the device resides in a subject's vagina. The devices may be used to treat or prevent a variety of biological conditions, including, but not limited to a sexually transmitted disease, pregnancy and a post-menopausal condition. The devices may be used to prevent or treat other biological conditions such as the bacterial, fungal, viral and/or protozoal infections disclosed in U.S. Pat. No. 6,591,654. In some embodiments, the biological condition is a sexually transmitted disease, including, but not limited to HIV. In some embodiments, the methods further comprise retainably positioning the intravaginal device within the vaginal tract of the subject. In further embodiments, the methods comprise retaining the intravaginal device in place for a period of time, including, but not limited to, about one day, about several days, about one month, or more than a month.

### EXAMPLES

The present invention is further illustrated by the following examples, which should not be construed as limiting in any way.

### Materials and Methods:

Medical-grade polyether urethane, Tecoflex® EG-80A, was provided by Noveon Inc., Thermedics Polymer Products (Wilmington, Mass.). Tecoflex® EG-80A is hereinafter referred to as PU. Dapivirine was provided by the International Partnership for Microbicides. Egg phosphotidyl choline (egg PC) was purchased from Acros (Morris Plains, N.J.). Epoxy parafilm paintable mold releasing agent was purchased from Price-Driscoll Corporate (Waterford, Conn.). All other chemicals were analytical or HPLC grade either purchased from Acros (Morris Plains, N.J.) or Aldrich (Milwaukee, Wis.).

HPLC quantification of dapivirine was conducted as follows. The HPLC system (Agilent 1050) consisted of a solvent module, quaternary pump module and a variable wavelength UV detector. For analysis of release and solubility study samples, an alkyl amide column (Supelcosil™ ABZ+Plus 3 μm, 3.3 cm×4.6 mm ID, Supelco, Bellefonte, Pa.) was used with a gradient of 50:50 acetonitrile and water to 100% acetonitrile (both containing 0.1% v/v trifluoroacetic acid, TFA) in 4 minutes at a flow rate of 1 mL/min. The retention time of dapivirine was 1.1 min. A linear calibration curve of dapivirine was obtained in the range of 1-100 ng/injection (r²>0.99) at the detection wavelength of 286 nm. The quantification limit of HPLC for dapivirine was 0.65 ng/injection (corresponds to 2 μl of a 1 μM solution) with a signal to noise ratio greater than 10. A longer method was developed for the stability study; a C-18 column (Hypersil BDS-C18, 10 cm×4.0 mm ID, 3 μM, Thermo Electron Corporation, Waltham, Mass.) was used with acetonitrile (A) and water (B) both containing 0.1% v/v TFA as eluent at a flow rate of 1 mL/min. A gradient of 30:70 A:B to 70:30 A:B was applied in 7 minutes with retention time of dapivirine at 5.7 minutes. The HPLC method was modified for LC/MS analysis: the flow rate was reduced to 0.5 mL/min; a gradient of 30:70 A:B to 70:30 A:B in 14 minutes; the TFA concentration in the eluents was 0.05% v/v and the retention time for dapivirine was 10.5 minutes.

### Example 1

### Formation of Drug Incorporated PU Rods and Rings

Incorporation of Dapivirine in PU matrix: Homogenous distribution of dapivirine in the PU matrix was achieved by two different methods. In the first method, PU and dapivirine (dapivirine/PU=20 mg/g, 10 mg/g, 5 mg/g, 2.5 mg/g) were dissolved in 75:25 mixture of dichloromethane and tetrahydrofuran. About 75% of the solvent was removed under reduced pressure and the viscous solution of PU and dapivirine was poured into a silanized crystallization flask which was further dried under air at a flow rate of 4 L/min for 2-3 days. The dapivirine incorporated PU films thus obtained were cut into small pieces and further dried under high vacuum until constant weight was achieved. In order to make the drug incorporation process simple and economically feasible for large scale production, the second method employed mixing of dapivirine crystals into molten PU. This method is closely related to the physical process of in-line screw compounding that would likely be used in the large scale production of rings made of PU thermoplastics. PU pellets were melted in a glass container heated to 150° C., dapivirine crystals were added (dapivirine/PU=20 mg/g and 2.5 mg/g) and mixed using an overhead stirrer at a speed of 20-30 rpm for 10 minutes followed by cooling to room temperature and processing into pellets by manually cutting into small pieces. The drug incorporated PU obtained by solvent casting and melt mixing are hereinafter referred to as SC and MM respectively. The dapivirine incorporated polymer pellets (both SC and MM) were optically clear, suggesting that dapivirine dissolved in the polymer matrix.

Extrusion of dapivirine loaded solid cross-sectional PU rods and formation of PU rings: Extrusion of SC and MM PU was conducted at 165-175° C. into solid cross-sectional tubes using a lab scale extruder (CSI Max Mixing Extruder CS-194A, Custom Scientific Instrument Inc., Easton, Pa.). The extruded and molten polymer output from the extruder was immediately fed to an aluminum mold (4.3 mm diameter, 25 cm length, previously spray coated with mold release agent) maintained at 120° C. The mold was cooled to below 80° C. and opened carefully without stretching the PU rod. The extruded PU rod was further cooled and washed with DI water. Extrusion of SC and MM pellets resulted in optically clear rods with slight yellowish tinge that increased with the dapivirine concentration, whereas the extruded rods from PU without dapivirine were clear and colorless. The extruded rods had a consistent diameter of 4.4±0.2 mm (n=12) throughout and weighed 162±5 mg/cm (n=24). Rings were fabricated by joining the ends of 17 cm lengths of extruded rods using molten PU, thus providing a ring of ID 54 mm and weight of 2.8±0.1 g (n=12).

### Example 2

### Stability of Dapivirine Under Extrusion Conditions

A series of experiments were conducted to confirm that dapivirine was stable in PU matrix in the extrusion process. First, the thermal stability of dapivirine at the extrusion temperature was examined and analyzed for its non-degradability as follows: dapivirine was heated at 185° C. isothermally for 2 h under air atmosphere and ¹H and ¹³C NMR spectra were recorded (Mercury 400 MHz spectrometer, Varian) for the heated and unheated dapivirine. Furthermore, LC/MS analysis was performed on the heated sample and compared to non-heated controls. The MS instrumentation consisted of Micromass Quattro II Triple Quadruple Mass Spectrometer, Waters, Milford, Mass. No change in the LC/MS and NMR spectra of the heated dapivirine sample was observed when compared with that of the non-heated sample indicating no detectable degradation of dapivirine.

Second, a differential scanning calorimetric (DSC) scan was obtained on dapivirine from 25° C. to 230° C. at a heating rate of 10° C./min under air atmosphere with a flow rate of 40 mL/min (Model DSC821e, Mettler Toledo, Columbus, Ohio). To further confirm the stability of dapivirine in the presence of PU under extrusion conditions, DSC analyses were carried out for PU, dapivirine, and PU films loaded with dapivirine at a concentration of 200 mg/g. The high concentration of dapivirine was selected to enable detection of potential exothermic degradation peaks. The DSC spectrum was collected from 25° C. to 230° C. at the heating rate of 10° C./min as well as at an isothermal condition (175° C. for 10 minutes).

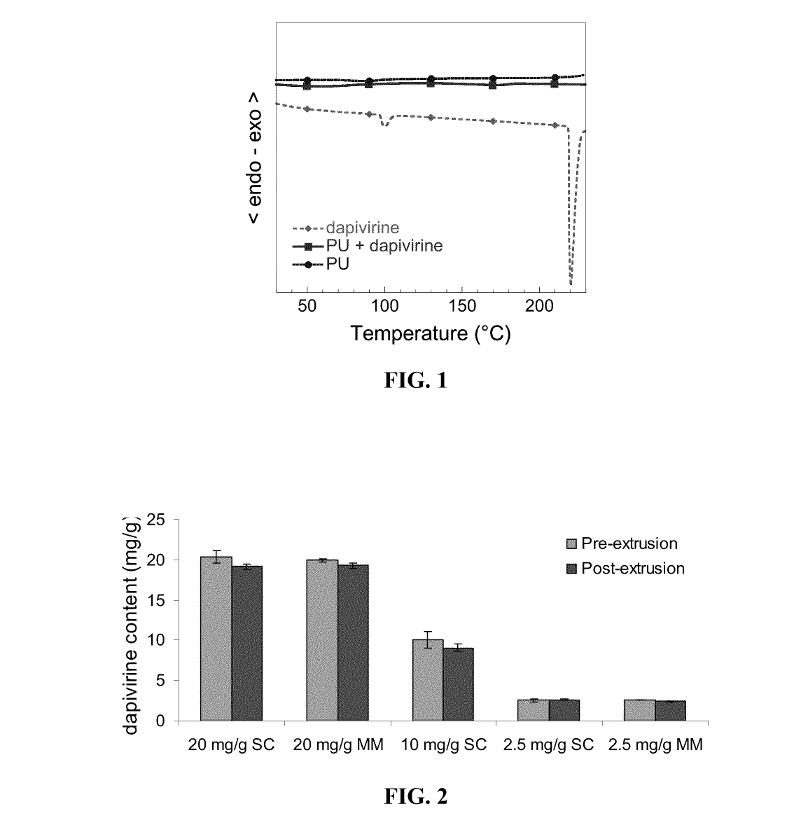

The results of the DSC experiments are shown in FIG. 1. The DSC thermogram of dapivirine from 25° C. to 230° C. showed no detectable degradation. The melting temperature of dapivirine was observed by an endotherm at 220° C. (the endothermic peak at 100° C. corresponds to moisture loss). Similarly, the DSC thermograms of PU and PU films loaded with dapivirine showed no detectable degradation. In particular, no exothermic peak was observed in the temperature ramp from 25 to 230° C. or in the isothermal step at 175° C. (data not shown) indicating no detectable degradation of dapivirine under extrusion conditions (165 to 175° C.). Also, absence of the endothermic peak at 220° C. corresponding to the melting point of dapivirine in the DSC spectrum of dapivirine loaded PU samples suggested a high solubility of dapivirine in PU matrix (>200 mg/g).

Finally, dapivirine was extracted and quantified from extruded rods and PU pellets (both SC and MM) prior to extrusion. The test samples (100 mg of ring segments or pellets) were dissolved in 2 mL of chloroform in a 50 mL centrifuge tube in triplicate followed by precipitation of the PU by addition of 25 mL of methanol using a volumetric flask. The polymer was separated by centrifugation (1600 RCF, 5 min) and the supernatant was quantified for dapivirine content by HPLC. The caps of the centrifuge tubes were closed tightly and wrapped with Teflon tape during the process to minimize evaporation. The efficiency of the extraction of dapivirine from the PU matrix using this method was determined by quantifying the extracted dapivirine content from controls of blank PU pellets (100 mg) to which known amount of dapivirine was added. The three controls used in triplicate were 1) 100 mg of PU pellets with 2 mg of dapivirine 2) 100 mg of PU pellets with 1 mg of dapivirine and 3) 100 mg of PU pellets with 0.25 mg of dapivirine. The mixtures of PU pellets and dapivirine were then subjected to the same precipitation process and dapivirine quantification as above. A linear calibration curve generated from standards of dapivirine in isopropanol (i-prOH) was utilized for quantification of dapivirine content from extracted samples.

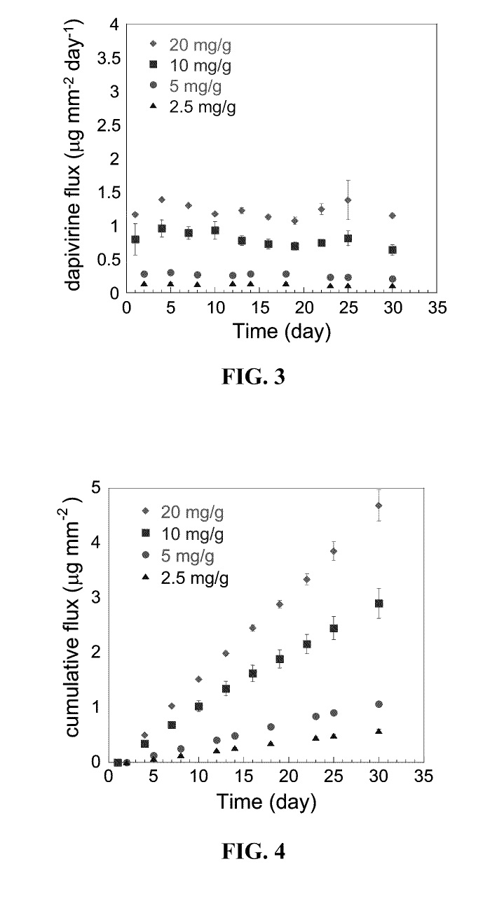

The extraction results are shown in FIG. 2. The efficiency of the extraction method was 100.1±4.6% as determined by the controls of blank PU pellets spiked with a known amount of dapivirine (3 individual experiments each with n=3 as specified in the methods section). The dapivirine contents recovered from extruded rods were not significantly different from those extracted from SC or MM pellets prior to extrusion (p value>0.05 using an unpaired student's t-test).

### Example 3

### Solubility Study of Dapivirine

Solubility studies of dapivirine were conducted to determine the appropriate sink conditions for the in vitro release studies. Dapivirine is a hydrophobic molecule; therefore a co-solvent system that can provide sink conditions (solubility greater than 3 times the maximum concentration achieved in the release medium) during dapivirine release study was needed. Since a co-solvent system consisting of 50:50 v/v i-prOH:water has been utilized previously for long-term release studies of dapivirine from silicone rings^{11,18}, the solubility of dapivirine was determined in 50:50 and 25:75 v/v i-prOH:water solutions. Additionally, solubility of dapivirine in liposome dispersions was evaluated in an attempt to utilize them as biorelevant sink conditions.^{14,21} The liposome dispersions of 10 mg/mL in 25 mM pH 4.2 acetate buffer (osmolarity adjusted to 310 mOsm/kg with NaCl) and 25 mM pH 7.6 phosphate buffer (osmolarity adjusted to 310 mOsm/kg with NaCl) were utilized. (See in vitro release studies in Example 4 for preparation of liposome dispersions.) Dapivirine (10 mg) was dispersed in test solutions (2 mL) and shaken at 37° C. at 200 rpm. Aliquots of 200 μL were withdrawn at 24 h, 48 h and 72 h. At each time point, the aliquots were centrifuged immediately for 2 minutes at 16000 g, 100 μL of the supernatant was diluted 1:10 using 100% isopropanol and quantified by HPLC. The solubility was determined by a calibration curve generated from known concentrations of dapivirine solutions in i-prOH.

The solubility data are shown in Table 1. From the solubility data, a 25:75 v/v i-prOH:water solution with a dapivirine solubility of 93 μM/L was used as the sink condition in drug release studies. Although a co-solvent system consisting of 50:50 v/v i-prOH:water has been utilized previously for long-term release studies of dapivirine from silicone rings^{11,18}, this system demonstrated up to 60% swelling of the PU matrix within a week. Such swelling will drastically impact the release kinetics of dapivirine from PU matrix. Therefore, the i-prOH ratio in the co-solvent system was reduced to 25% v/v. The measured dapivirine solubility in 25:75 v/v i-prOH:water co-solvent system at 37° C. was higher than 3 times the maximum dapivirine concentration observed in the maximum loading (20 mg/g) thereby satisfying sink conditions during the release study.

TABLE 1
Solubility of dapivirine

| | Dapivirine solubility at | | Time to reach |
|---|---|---|---|
| Solvent | 37° C. (μM) | | saturation |
| 25:75 i-prOH:water | 93 | μM | 48 h |
| 50:50 i-prOH:water | 3306 | μM | 24 h |
| 10 mg/mL pH 4.2 liposome | 844 | μM | 48 h |
| dispersion | | | |
| 10 mg/mL pH 7.6 liposome | 165 | μM | 48 h |
| dispersion | | | |

### Example 4

### In Vitro Release Studies of Dapivirine Loaded PU Rods and Rings

The release profile of dapivirine was measured from PU-SC rods for a month after which the release kinetics from rings was evaluated for two weeks to determine the correlation of drug release rate from rods to that from rings. This may enable use of rods in place of rings for release kinetics evaluation thereby minimizing the scale of materials such as ring materials, quantity of drugs and volume of sink solutions required for release studies in case of monolithic IVRs. About 2 cm length segments were cut from the solid cross-sectional dapivirine loaded PU SC rods. The dimensions (length and diameter) and weight of each segment were recorded. The segments were incubated with 5 mL 25:75 v/v i-prOH:water as the sink condition in 20 mL septa covered vials at 37° C. in a water bath shaker set at 64±2 rpm. The sink was replaced with fresh co-solvent every 24±0.5 h for 30 days. HPLC analyses were performed every 2 to 5 days and the cumulative release was calculated by integrating the area under the curve using Kaleidagraph software (Synergy Software, Reading, Pa.). The release study for each loading was performed on triplicate samples. At the end of 30 days, the segments were wiped dry and the masses and dimensions of the segments were recorded. Similarly, dapivirine release kinetics were measured from rings (20 mg/g SC, 2.5 mg/g SC, 20 mg/g MM and 2.5 mg/g MM) incubated with 50 mL of 25:75 v/v i-prOH:water mixture as sink condition in 125 mL Erlenmeyer flasks with rubber septa for a month.

At the end of each release study, the swelling ratio was calculated by using the following equation

$$Q = \frac{( {M}_{f} - {M}_{i} )}{{M}_{i}}$$

where
Q=swelling ratio
Mᵢ=mass of ring or rod segments before the release study
M_{f}=mass ring or rod segments after the release study

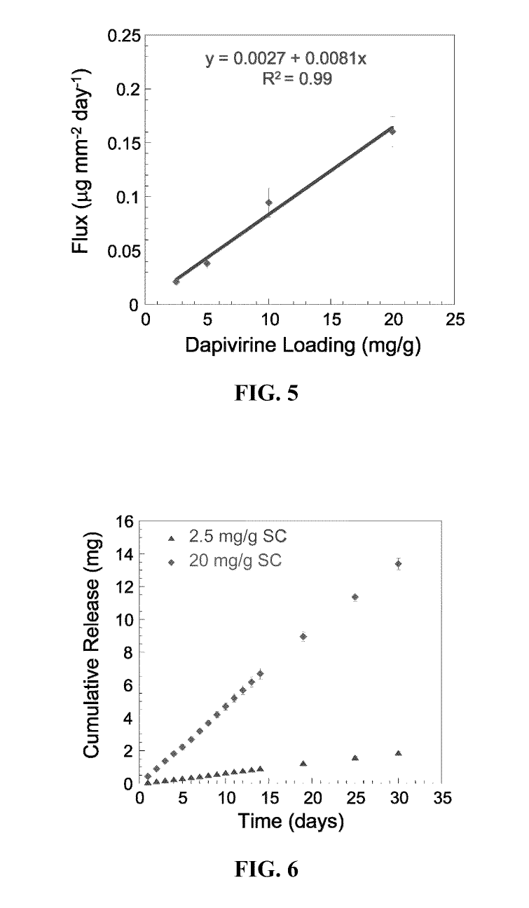

The results of the rod studies are shown in FIGS. 3-5. As shown in FIG. 3, a sustained release of dapivirine was observed under 25:75 i-prOH:water from all of the drug loadings tested. As shown in FIG. 4, the cumulative flux with respect to time revealed a linear relationship with r² value greater than 0.99 for all loadings, and thereby a zero order release kinetics for a month. At the end of 30 days, about 20% of the loaded dapivirine was released and there was 15±3.7% (n=12) swelling observed in the rods irrespective of the loading. As shown in FIG. 5, the dapivirine flux obtained illustrated a direct proportionality to the dapivirine loaded concentration.

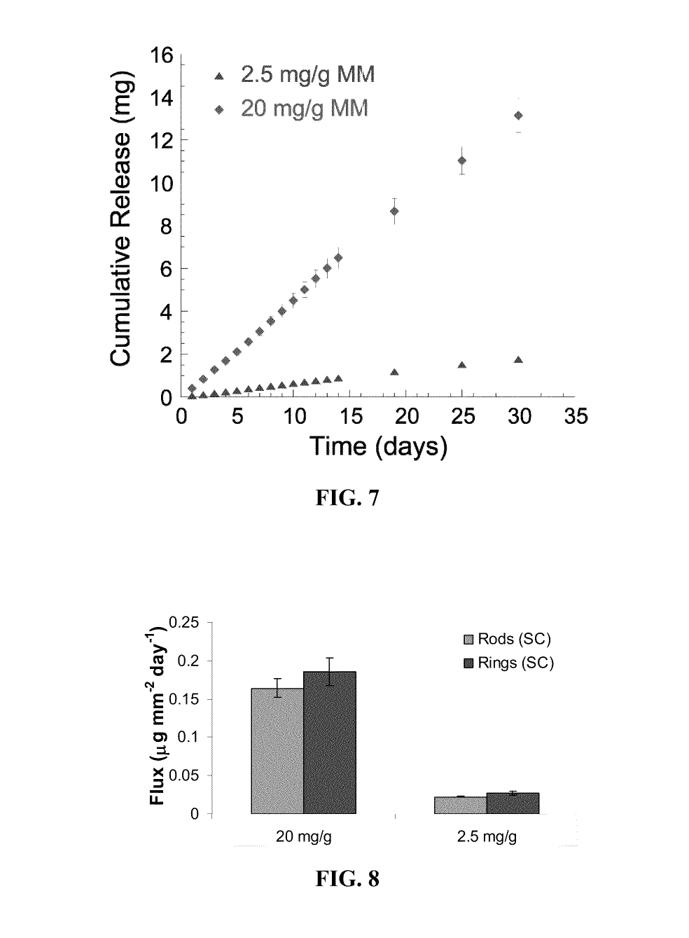

The results of the ring studies are shown in FIGS. 6-7. As shown in these figures, the cumulative dapivirine release obtained was linear with time verifying a zero order release rate. There was no significant difference (p>0.05, unpaired student's two tailed t-test) between the release kinetics obtained from rings constructed by the two different processing methods (SC and MM). The average release rates in two weeks duration from 2.5 mg/g and 20 mg/g dapivirine loadings were 64±1 μg/day and 473±36 μg/day, respectively. As shown in FIG. 8, the average fluxes obtained in 20 mg/g SC and 2.5 mg/g SC rings were 12 and 18% higher, respectively, than those from rods. This may be due to differences in the geometry of a solid cylinder with open ends versus a torus shape of a ring and/or due to the slightly higher ratio of volume of sink used to the length of dapivirine loaded segments. The ratio of volume of 25:75 v/v i-prOH:water sink used per unit cm length of dapivirine loaded PU segment was 17.5% higher in the release study with rings compared to that in the release study from rod segments. The average swelling observed at the end of one month period in the rings was 19.4±0.4% respectively (n=12).

It is believed that vaginally delivered lipophilic NNRTI's partition into the lipid bilayer of vaginal epithelial cells.^{14,21} Therefore, additional release studies were performed using 100 nm liposome dispersions in pH 4.2 and pH 7.6 buffers as biorelevant sink conditions for 1 week. These pH conditions cover the pH range observed vaginally. Liposome dispersions were synthesized by dissolving egg PC in CHCl₃ followed by solvent removal under vacuum. The dried egg PC cake was dispersed in either pH 4.2 (25 mM acetate buffer containing 0.02% sodium azide and osmolarity adjusted to 310 mOsm/kg with NaCl) or pH 7.6 (25 mM phosphate buffer containing 0.02% sodium azide and osmolarity adjusted to 310 mOsm/kg with NaCl) followed by extrusion through a 100 nm membrane (LiposoFast™, Avestin, Ottawa, Canada). The concentration of liposome was 10 mg/mL. Prior to conducting the release study, liposome dispersions were characterized for particle size and zeta potential in a Zetasizer (nano ZS90, Malvern Instruments, Malvern Worcestershire, UK). A third sink condition for comparison was 25:75 v/v i-prOH:water. Approximately 0.5 mm length segments of dapivirine loaded (5 mg/g) PU rods were incubated with 0.5 mL of sink solution in 37° C. water bath shaking at 64±2 rpm. Controls used were dapivirine loaded PU rods suspended with pH 4.2 and pH 7.6 buffers without liposomes and blank PU (no dapivirine) segments in liposome dispersions in pH 4.2 and pH 7.6 buffers. Sinks were replaced and quantified for dapivirine content at frequent time points. HPLC quantification of release in sink samples of liposome dispersions was performed after 1:1 dilution with i-prOH to disrupt the dispersion.

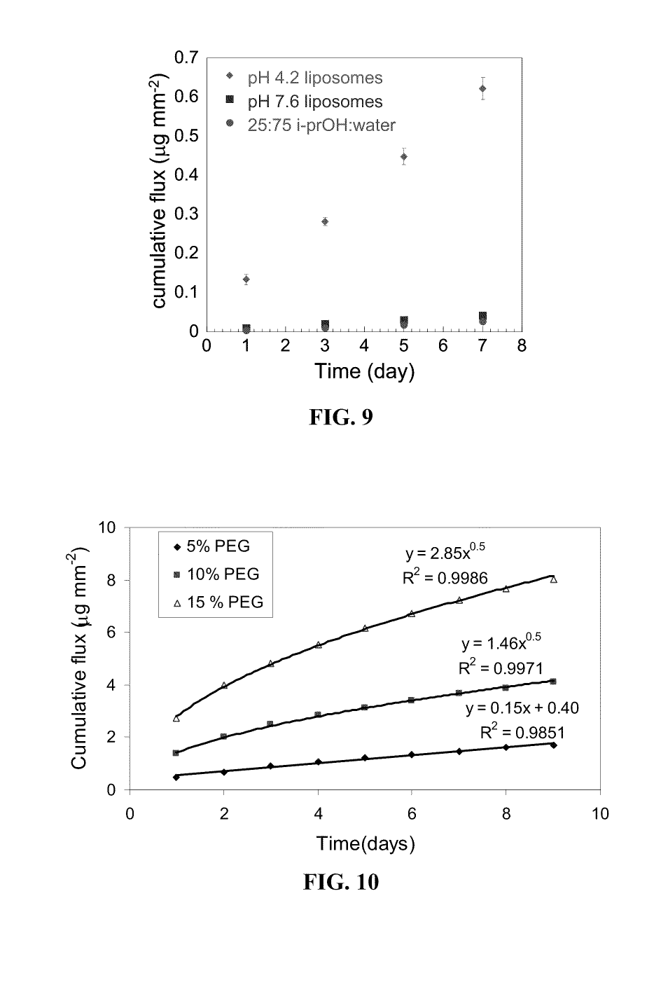

The liposome dispersion in pH 7.6 buffer demonstrated a particle size of 135 nm with polydispersity of 0.118 and a zeta potential of −25.1. The liposome dispersion in pH 4.2 buffer demonstrated a particle size of 279 nm with polydispersity of 0.223 and a zeta potential of −20.2. As shown in FIG. 9, the plot of cumulative release of dapivirine in liposome dispersions over time showed a linear relationship with time for the one week period tested. The dapivirine release in buffer controls without liposomes was below the detection limit of the method. The release kinetics from the same drug loading (5 mg/g) under similar conditions in pH 4.2 and pH 7.6 liposome dispersions were 22 times and 1.5 times, respectively, of those observed in the 25:75 i-prOH:water sink condition, as calculated by comparing the slopes of the linear curve fits. The swelling of the dapivirine loaded PU rods (5 mg/g) in liposome dispersions was 2.6±0.7% (n=6). The increased release rate in acidic liposome dispersions suggests that hydrophobic dapivirine may readily partition into the lipid bilayer of vaginal epithelial cells. It is postulated that the lower charge density of pH 4.2 liposomes⁴² improved the partitioning of the hydrophobic dapivirine molecules into the liposomes.

### Example 5

### In Vitro Release Studies of Adenosine Monophosphate (AMP), a Model Compound for Tenofovir, from PU Rings Containing Polyethylene Glycol of Mn 2000 (PEG2000). (Mn=Number Average Molecular Weight)

(Tenofovir used in all the examples below was provided by International Partnership for Microbicides. PEG2000 used in all the examples below was obtained from Aldrich)

Incorporation of AMP in PU matrix: Homogenous distribution of AMP and PEG2000 in the PU matrix was achieved by solvent casting. AMP (500 mg), PEG2000 (500 mg (5% w/w), 1000 mg (10% w/w) and 1500 mg (15% w/w)) and PU (added to make the total weight of PEG+AMP+PU mixture to 10 g) were dissolved in 75:25 mixture of dichloromethane and tetrahydrofuran. About 75% of the solvent was removed under reduced pressure and the viscous solution of PU, AMP and PEG2000 was poured into a silanized crystallization flask which was further dried under air at a flow rate of 4 L/min for 2-3 days. The Tenofovir incorporated PU films thus obtained were cut into small pieces and further dried under high vacuum until constant weight was achieved.

Extrusion of PEG2000 and AMP loaded solid cross-sectional PU: Extrusion was conducted at 165-175° C. into solid cross-sectional rods using a lab scale extruder (CSI Max Mixing Extruder CS-194A, Custom Scientific Instrument Inc., Easton, Pa.). The extruded and molten polymer output from the extruder was immediately fed to an aluminum mold (4.3 mm diameter, 25 cm length, previously spray coated with mold release agent) maintained at 120° C. The mold was cooled to below 80° C. and opened carefully without stretching the PU rod. The release profile of AMP was measured from PU rods using pH 4.2 25 mM acetate buffer (osmolarity adjusted to 310 mOsm/kg using NaCl, 0.02% sodium azide) at 37° C. and 60 rpm. About 2 cm length segments were cut from the solid cross-sectional AMP loaded PU rods. The dimensions (length and diameter) and weight of each segment were recorded. The sink was replaced with fresh buffer every 24±0.5 h for 10 days. HPLC analyses were performed and the cumulative release was calculated by integrating the area under the curve using Kaleidagraph software (Synergy Software, Reading, Pa.). The release study for each loading was performed on triplicate samples. The release profiles, shown in FIG. 10, demonstrate that PEG incorporation changes the release kinetics and increases the overall amount of drug released per day. A burst release is obtained in all PEG-PU compositions that is proportional to the PEG concentration. The release profile is diffusion limited in rings containing 10% and 15% w/w PEG, but zero order in rings containing 5% w/w PEG.

### Example 6

### Designs for IVRs

One design for an IVR is shown in FIG. 11. Different compartments of drug loaded PUs are made by separately extruding the different drug loaded PU and joining segments from the separately extruded drug loaded PU using a biocompatible adhesive, such as the molten PU itself. The two compartments can also be separated by using a placebo compartment of non-medicated PU.

Another design for an IVR is shown in FIG. 12. The ring comprises an inner, non-medicated core of one PU (PU 1), surrounded by an outer layer of a different, medicated PU (PU 2). The composition of PU 2 can be optimized by adjusting the hard/soft segment ratio of the PU, thereby controlling and tailoring the release kinetics of the incorporated drugs. A nonmedicated and hard inner PU 1 core acts as a support. Alternatively, the inner core can be coated with one or more compartments of different drug loaded soft PU 2.

### Example 7

### Fabrication of Tenofovir Loaded Tecophilic Matrices and Release Profile of Tenofovir from Tecophilic Matrix

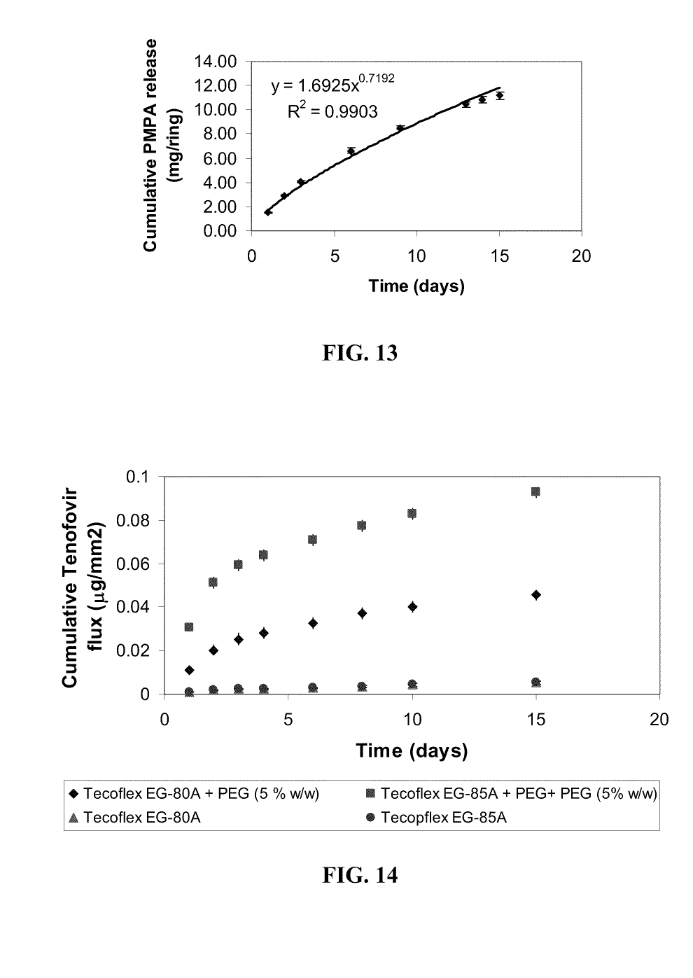

Sample Preparation: 10.6 g of Tecophilic HP-60D-20 pellets were mixed with 508 mg, of Tenofovir until the polymer pellets were uniformly coated with tenofovir powder by qualitative observation. The mixture was then extruded at 165-175° C. without any mold. The extruded rod was whitish and turbid in color (extruded Tecophilic without Tenofovir was colorless and transparent). The actual Tenofovir content in the rods has not been determined. Rods with average diameter of 3.72±0.16 and about 2 cm in length were used for conducting release studies. Rods were incubated with 25 mM pH 4.2 acetate buffer (osmolarity adjusted to 310 mOsm with NaCl, 0.02% sodium azide) at 37° C. and 60 rpm. The release media was changed every 24±0.5 hrs. The release study was conduced on triplicate samples. The results are shown in FIG. 13.

### Example 8

### Fabrication and Release Study of Tenofovir from Tecoflex EG-80A and Tecoflex EG-85A Matrices with and without PEG 2000

Incorporation of Tenofovir and PEG2000 in Tecoflex EG-80A and EG-85A matrices: Homogenous distribution of Tenofovir and PEG2000 in Tecoflex EG-80A or EG-85A_matrices were achieved by solvent casting. Tenofovir (500 mg) with or without PEG2000 (500 mg) were dissolved in 75:25 mixture of dichloromethane and tetrahydrofuran. About 75% of the solvent was removed under reduced pressure and the viscous solution was poured into a silanized crystallization flask which was further dried under air at a flow rate of 4 L/min for 2-3 days. The Tenofovir incorporated polymer films thus obtained were cut into small pieces and further dried under high vacuum until constant weight was achieved.

Solid cross-sectional rods were obtained by extrusion of polymer films obtained in the above step at 155-165° C. using a lab scale extruder (CSI Max Mixing Extruder CS-194A, Custom Scientific Instrument Inc., Easton, Pa.). Small rings were fabricated by joining the ends of rods of approximately 6 cm in length. The release profile of Tenofovir was measured from Tenofovir loaded small rings using pH 4.2 25 mM acetate buffer (osmolarity adjusted to 310 mOsm/kg using NaCl, 0.02% sodium azide) at 37° C. and 60 rpm. The sink was replaced with fresh buffer every 24±0.5 h for 15 days. HPLC analyses were performed and the cumulative release was calculated by integrating the area under the curve using Kaleidagraph software (Synergy Software, Reading, Pa.). The release study for each loading was performed on triplicate samples. The release profiles, shown in FIG. 14, demonstrate that PEG incorporation changes the release kinetics and increases the overall amount of drug released per day from both Tecoflex EG-80A and Tecoflex EG-85A matrices.

### Example 9

### Fabrication and Release Study of Tenofovir from Tecoflex EG-93A Matrices with PEG2000 and with and without the Presence of Salts

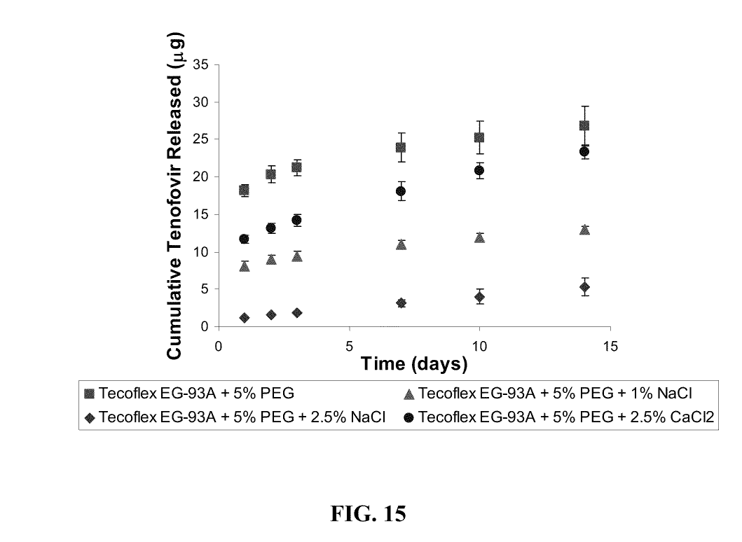

Incorporation of Tenofovir and PEG2000 and salts in EG-93A matrices: Homogenous distribution of Tenofovir, PEG2000 with and without salts in Tecoflex EG-93A_matrices were achieved by solvent casting. Tenofovir (250 mg), PEG2000 (250 mg (5% w/w)) and salts (NaCl=50 mg (1% w/w) or 125 mg (2.5% w/w) or CaCl₂=125 mg (2.5% w/w)) were dispersed in 75:25 mixture of dichloromethane and tetrahydrofuran followed by sonication for 30 minutes. About 75% of the solvent was removed under reduced pressure and the viscous solution was poured into a silanized crystallization flask which was further dried under air at a flow rate of 4 L/min for 2-3 days. The Tenofovir incorporated polymer films thus obtained were cut into small pieces and further dried under high vacuum until constant weight was achieved. About 1.2 g of the polymer film pieces were taken in a 20 mL scintillation vial which was incubated at 210° C. for about 5-6 minutes to allow the polymer pieces to melt and fuse to form a continuous film in the bottom of the vial. Then 5 mL of sink (25 mM pH 4.2 buffer, osmolarity adjusted to 310 mOsm/kg with NaCl, and 0.02% sodium azide) was added. The sink was replaced with fresh buffer every 24±0.5 h for 15 days. HPLC analyses were performed and the cumulative release was calculated by integrating the area under the curve using Kaleidagraph software (Synergy Software, Reading, Pa.). The release study for each loading was performed on triplicate samples. The release profiles, shown in FIG. 15, demonstrate that incorporation of salts changes the release kinetics of Tenofovir from Tecoflex EG-93A matrices. The incorporation of 2.5 w/w NaCl or CaCl₂ produced a nearly zero-order release of Tenofovir from Tecoflex EG-93A matrix.

### Example 10

### Fabrication and Release Study of Various Drugs from Tecoflex EG-80A, Tecoflex EG-93A Matrices, and Tecophilic HP-60D-20

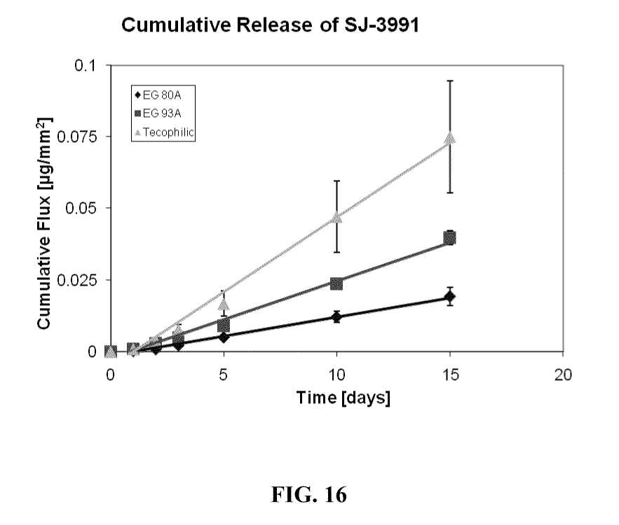

The antiviral drugs SJ-3366 (1-(cyclopent-3-enylmethyl)-6-(3,5-dimethylbenzoyl)-5-ethylpyrimidine-2,4(1H,3H)-dione), SJ-4010 (1-(cyclopentenylmethyl)-6-(3,5-dimethylbenzoyl)-5-isopropylpyrimidine-2,4(1H,3H)-dione), SJ-3339 (1-(cyclopent-3-enylmethyl)-6-(3,5-dimethylbenzoyl)-5-isopropylpyrimidine-2,4(1H,3H)-dione), SJ-3991 (1-(cyclopropylmethyl)-6-(3,5-dimethylbenzoyl)-5-isopropylpyrimidine-2,4(1H,3H)-dione), BMS 793 (1-(4-benzoyl-2,2-dimethylpiperazin-1-yl)-2-(3H-pyrrolo[2,3-b]pyridin-3-yl)ethane-1,2-dione), and the contraceptive steroid 19-norethindrone were used in this example. 1 g of Tecoflex EG-80A, Tecoflex EG-93A or Tecophilic HP-60D-20 was dissolved in 10 mL of THF (tetrahydrofuran) and placed in a tarred 20 mL scintillation vial and was shaken at 250 rpm at 25° C. or 60° C. From a concentrated stock solution of the drug in THF, 5 mg of drug substance was added into each of the scintillation vials containing the dissolved polymer. All samples were prepared in triplicates. The drug/polymer formulation was allowed to shake for 2 hours at 25° C. for complete mixing and the solvent was evaporated under vacuum until the sample obtained a constant mass. Excess film was cut from the sides so the film was a circular disk. The final mass of the sample in the scintillation vial was ˜0.5 g. A solution of 2% Labrasol (pH 4.2 acetate buffer) was placed in the vial and shaken at 80 rpm at 37° C. The samples were removed each day at the same time, and a fresh 2% Labrasol at pH 4.2 was added. The solvent was collected in HPLC vials on day 1, 2, 3, 5, 10 and 15 and stored at −80° C. The drug concentration of the samples was evaluated by HPLC performed on an Agilent 1200 Series HPLC equipped with ChemStation 32 software. The samples were analyzed on a Supelcosil™ ABZbPlus 3 mm, 3.3 cm_{—}4.6 mm ID, Supelco at a column temperature of 25° C. at 267 nm and 280 nm. Mobile Phase A was HPLC grade Acetonitrile (0.1% v/v trifluoroacetic acid) and mobile phase B was 18 mΩ water (0.1% v/v trifluoroacetic acid). The flow rate was 0.5 mL/min with a gradient of 0% A to 100% B over 17 minutes. All samples showed zero order release from the polyurethane composition. FIG. 16 shows the results for the antiviral drug SJ-3991.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

For the purposes of this disclosure and unless otherwise specified, “a” or “an” means “one or more.” All patents, applications, references and publications cited herein are incorporated by reference in their entirety to the same extent as if they were individually incorporated by reference.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

1. An intravaginal device comprising a polyether urethane composition and a pharmaceutically effective amount of at least one vaginally administrable drug homogeneously distributed throughout the polyether urethane composition.

2. The device of claim 1, wherein the polyether urethane composition comprises a Tecoflex® polyurethane.

3. The device of claim 2, wherein the Tecoflex® polyurethane is selected from Tecoflex® EG-80A, Tecoflex® EG-85A, or Tecoflex® EG-93A polyurethane.

4. The device of claim 1, wherein the polyether urethane composition comprises a Tecophilic® polyurethane.

5. The device of claim 4, wherein the Tecophilic polyurethane is Tecophilic® HP-60D-20 polyurethane.

6. The device of claim 1, wherein the polyether urethane composition comprises a polyurethane formed from the reaction of a diisocyanate, a polymeric diol, and a short chain diol.

7. The device of claim 1, wherein the polyether urethane composition comprises a polyurethane formed from the reaction of dicyclohexyl methane diisocyanate, a polytetramethylene ether polyol having a molecular weight of between about 500 and about 10,000, and 1,4 butane diol.

8. The device of claim 1, wherein the drug is selected from the group consisting of microbicides, contraceptive agents, hormones, estrogen receptor modulators, post-menopausal hormones, antiviral agents, anticancer agents, and therapeutic peptides.

9. The device of claim 8, wherein the drug is a microbicide and the microbicide is an anti-HIV or an anti-HPV agent.

10. The device of claim 8, wherein the drug is an anti-HIV agent selected from the group consisting of non-nucleoside reverse transcriptase inhibitors, nucleoside reverse transcriptase inhibitors, and HIV entry inhibitors.

11. The device of claim 10, wherein the non-nucleoside reverse transcriptase inhibitor is dapivirine and the nucleoside reverse transcriptase inhibitor is Tenofovir.

12. The device of claim 8, wherein the drug is selected from 1-(cyclopent-3-enylmethyl)-6-(3,5-dimethylbenzoyl)-5-ethylpyrimidine-2,4(1H,3H)-dione, 1-(cyclopentenylmethyl)-6-(3,5-dimethylbenzoyl)-5-isopropylpyrimidine-2,4(1H,3H)-dione, 1-(cyclopent-3-enylmethyl)-6-(3,5-dimethylbenzoyl)-5-isopropylpyrimidine-2,4(1H,3H)-dione, 1-(cyclopropylmethyl)-6-(3,5-dimethylbenzoyl)-5-isopropylpyrimidine-2,4(1H,3H)-dione, 1-(4-benzoyl-2,2-dimethylpiperazin-1-yl)-2-(3H-pyrrolo[2,3-b]pyridin-3-yl)ethane-1,2-dione, or 19-norethindrone.

13. The device of claim 1, wherein the drug is present in an amount ranging from about 2 mg to about 60 mg of drug per gram of polyether urethane.

14. The device of claim 1, wherein the drug is present in an amount ranging from about 0.1% w/w to about 10% w/w.

15. The device of claim 1, further comprising polyethylene glycol incorporated into the polyether urethane matrix.

16. The device of claim 15, wherein the polyethylene glycol is present in an amount ranging from about 5% w/w to about 15% w/w.

17. The device of claim 1, wherein the device exhibits a substantially zero order release profile of the drug over a period of at least one day.

18. The device of claim 1, wherein the device exhibits a release rate of drug ranging from about 55 μg of drug per day to about 550 μg of drug per day.

19. The device of claim 1, wherein the intravaginal device is an intravaginal ring.

20. A method of making an intravaginal device comprising forming a drug-loaded polyether urethane composition into a shape suitable for use in intravaginal drug delivery, wherein a pharmaceutically effective amount of at least one vaginally administrable drug is homogeneously distributed throughout the composition.

21-23. (canceled)

24. A method comprising releasing the drug from the intravaginal device of claim 1 while the device resides in a subject's vagina.

25-26. (canceled)
